(19) United States
(12) Patent Application Publication
(10) Pub. No.: US 2002/0137097 A1
(43) Pub. Date: Sep. 26, 2002

(54) Standard diluent for multiplex assays

(75) Inventors: Quan Nguyen, Pleasant Hill, CA (US); Aiguo Zhang, Hayward, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA

(21) Appl. No.: 10/017,788

(22) Filed: Dec. 13, 2001

Related U.S. Application Data
(60) Provisional application No. 60/255,561, filed on Dec. 13, 2000.

(51) Int. Cl. G01N 33/53; G01N 33/543

(52) U.S. Cl. 435/7.1; 436/518

(57) ABSTRACT

The present invention provides standard diluents for use in multiplex assays comprising a biological fluid that normally includes two or more different target analytes but that is substantially free of the two or more different target analytes, as well as kits and methods relating to the standard diluent. The standard diluents of the invention provide an accurate and reliable means to calibrate the amount of multiple target analytes in a single assay.

### BACKGROUND OF THE INVENTION

A standard diluent is a key component of immunoassays. It is used as a buffer for diluting a reference standard that is used to quantify target analytes in a test sample. Generally, to quantify the amount of target analytes present in a test sample, a series of control materials containing different concentrations of the target analytes are prepared. The series of control materials are then used to prepare a standard curve with the concentration of the target analyte plotted on one axis and a detection signal strength on the other axis. The concentration of the target analyte present in a test sample can be interpolated from such a standard curve.

In order for the quantitation of target analytes in a test sample to be accurate, the target analyte must behave similarly in the standard diluent and in the test sample. Sometimes components present in the test sample or in the standard diluent may influence binding of the target analyte to its antibodies. For example, some unknown components present in a standard diluent may bind to the target analytes and reduce their binding to antibodies. Such unknown components may not be present in the test sample. In such an instance, the concentration of the target analytes in the test sample interpolated from the standard curve generated using the standard diluent would be higher than the actual concentration of the target analytes in the test sample because of the interaction between the target analytes and the unknown components. Typically, a standard diluent is selected so that the standard curve generated using the standard diluent and the test sample deviates no more than ±20%. This is commonly described as a recovery assay by immunoassay developers.

For the detection of a single analyte, it is relatively easy to develop a standard diluent since the standard diluent can be readily optimized to mimic only a single antibody-analyte reaction in a test sample. A standard diluent is generally formulated to provide reaction conditions and an environment that are equivalent to those encountered in the biological test sample to be assayed. Typically, an artificial cocktail of proteins, buffers and salt is used as a standard diluent that can duplicate the condition of a test sample to detect a single analyte. However, different target analytes require different conditions, and an artificial cocktail that is suitable for analysis of one target analyte may not be suitable for analysis of another target analyte.

In recent years, methods have been developed to simultaneously detect multiple different analytes in a single assay process (e.g., in a single well). Such methods are generally known by terms such as “multiplex assays” or “multiplex immunoassays.” In a multiplex immunoassay, selecting a standard diluent that is suitable for all of the different analytes is more complex, since each antibody-analyte interaction operates best under its own set of conditions. Thus, a standard diluent comprising an artificial cocktail optimized to duplicate the condition of a test sample for one analyte may not be optimal for the detection and quantitation of other analytes. Hence, developing an artificial standard diluent that is suitable for multiplex assay is difficult and time-consuming, if not impossible. In particular when the number of target analytes to be simultaneously detected increases to, for example, fifteen to twenty, it is difficult to develop a standard diluent that provides the appropriate operating environment for all of the target analytes at the same time.

Accordingly, there is a need to develop a standard diluent for multiplex assays so that two or more different target analytes can be simultaneously detected and reliably quantified. Embodiments of the invention address this and other needs.

### SUMMARY OF THE INVENTION

It has now been discovered that a standard diluent for multiplex immunoassays can be derived from a biological fluid that normally contains two or more different target analytes to be detected in an immunoassay, but is processed or screened to be substantially free of these target analytes. For example, if the simultaneous detection of four types of cytokines in a human serum sample is desired, a standard diluent used to dilute a reference standard to quantify cytokines is derived from a human serum that is substantially free of the four types of cytokines. A standard diluent “substantially free of two or more different target analytes” means that the endogenous level of target analytes normally present in the biological fluid is no longer detectable in the standard diluent or that the amount of target analytes present in the biological fluid is below a selected threshold level (e.g., 10 pg/mL). Typically, the endogenous level of target analytes is removed from the biological fluid by affinity chromatography. Alternatively, biological fluids obtained from donors can be screened to identify a biological fluid that naturally contains the target analytes in amounts that are not detectable, or that are below a selected threshold level. Such processed or screened biological fluids comprise essentially the same matrix components as a sample to be tested, and are ideally suited for use as a standard diluent. By providing essentially the same matrix components as a test sample, the standard diluent of the invention provides reaction conditions and an environment for multiple target analytes that are equivalent to the biological test sample. These qualities enable the standard diluent of this invention to facilitate reliable and accurate quantitation of target analytes in a biological sample.

The standard diluent of the present invention has utilities in various multiplex assays, particularly when quantitation of target analytes needs to be accurate. For example, the standard diluent can be used in multiplex assays for monitoring the amount of target analytes in a clinical sample. For instance, the target analytes can be components of an immune system (e.g., cytokines) in patients who are suffering from autoimmune diseases or cancer and whose immune system needs to be evaluated periodically to determine progress of their disease. Alternatively, the target analytes can be various proteins that are associated with a certain disease. For instance, multiple proteins, including prostate cancer antigen, are known to be elevated in patients having prostate cancer. A standard diluent that is substantially free of prostate cancer antigen and other proteins associated with prostate cancer can be used to accurately determine the concentration of these proteins in a clinical sample. In another example, the standard diluent can also be used in a drug discovery program in determining the concentration of certain target analytes that are stimulated by a test drug.

Accordingly, in one aspect the invention provides a standard diluent comprising a biological fluid normally including two or more different target analytes but that has been processed or screened to be substantially free of the two or more different target analytes. In a preferred embodiment, the standard diluent is derived from serum or plasma, and the target analytes are cytokines.

In another aspect, the invention provides a kit comprising: (a) a standard diluent comprising a biological fluid normally including two or more different target analytes but that is substantially free of the two or more different target analytes; and (b) a predetermined amount of one or more concentrated materials that collectively or separately contain the two or more different target analytes. Such a kit can be used to make a series of control materials containing different concentrations of target analytes to generate a standard curve. The standard curve can then be used to quantify the amount of target analytes present in test samples.

In another aspect, the invention provides a kit for detecting two or more different target analytes in a serum or plasma sample, the kit comprising: (a) solid supports that are classifiable into subgroups, each subgroup differentiable from others by a differentiation parameter and each subgroup capable of having immobilized thereon a capture reagent that binds to a particular target analyte; and (b) a standard diluent comprising serum or plasma that is substantially free of the two or more different target analytes. Such a kit can be used to simultaneously detect and quantify multiple target analytes from a serum or plasma sample.

In another aspect, the invention comprises a method of conducting a simultaneous assay for two or more target analytes in which a standard diluent is used to dilute one or more reference standards, the method comprising using as the standard diluent a biological fluid that is substantially free of the two or more target analytes.

In a further aspect, the invention comprises a method of preparing a standard diluent for use in a simultaneous assay for two or more target analytes, comprising treating a biological fluid containing the target analytes to remove the target analytes so as to decrease the concentrations thereof to concentrations below predetermined thresholds.

These and other features, objects, and advantages of the invention will be more readily understandable from the description that follows.

### BRIEF DESCRIPTION OF THE DRAWINGS

FIG. 1 illustrates the amount of eight cytokines measured simultaneously in serum samples obtained from eleven patients.

FIG. 2 illustrates a recovery study comparing standard diluent and serum samples in three human patients.

FIG. 3 illustrates standard curves from a simultaneous determination of the same eight cytokines as in FIG. 1, in mouse sera.

FIG. 4 illustrates a mouse sera recovery study in which serum samples were taken from two lots of mice.

### DESCRIPTION OF SPECIFIC EMBODIMENTS

In one aspect, the invention provides a standard diluent comprising a biological fluid that normally includes two or more different target analytes but that in the present case is substantially free of these analytes. The selection of a biological fluid to make a standard diluent depends on the source of biological fluid in which the detection of the target analytes is desired. By “the source” it is not meant to refer to a specific individual but rather to a general type of biological fluid from a human or from a type or species of animal. For example, if the detection of various cytokines in blood serum is desired to monitor a subject's immune response, then a blood serum is considered to be the source from which to make a standard diluent. Hence, a standard diluent can be derived from various types of biological fluids depending on the selection of a test sample for the detection of target analytes. For example, the biological fluid in which the target analytes are sought can be a serum, plasma, urine, cerebrospinal fluid, tissue or cell extract, amniotic fluid, sweat, tear, saliva or nasal secretion, and, correspondingly, the standard diluent is prepared from the same type of fluid or source. The biological fluid can be obtained from either a human or a non-human (e.g., mouse, rat, guinea pig, rabbit, etc.); the fluid need not be obtained from the same species as that whose fluid is to be analyzed for the targets.

To make a standard diluent substantially free of two or more different target analytes, the target analytes are typically removed from the biological fluid. There are a number of known suitable techniques that can be used to remove the target analytes. A preferred technique for this purpose is affinity chromatography. For example, column chromatography techniques can be applied to remove the target analytes and to obtain an eluent substantially free of the target analytes for use as a standard diluent. Any suitable adsorbents can be used, as long as they are capable of selectively removing the target analytes from the biological fluid. It is preferred that antibodies that specifically bind to the target analytes are employed to remove the target analytes from the biological fluid. The term “antibodies” include monoclonal antibodies, polyclonal antibodies, antibody fragments, single chain antibodies, etc. Methods for making antibodies are described in detail below. The antibodies in question can be conjugated to column matrices. A biological fluid can be applied to the column, and an eluent from which the target analytes have been removed may be collected and used as a standard diluent. The column chromatography and other affinity chromatography methods are well known in the art and are described in, e.g., Scope, Protein Purification, Principles and Practice, 3rd ed., Springer-Verlag New York, Inc. (1994); and Deutscher, Methods in Enzymology: Guide to Protein Purification, Vol. 182, Academic Press, San Diego (1990).

Alternatively, a standard diluent substantially free of two or more different target analytes may be obtained by screening biological fluids from various sources that naturally do not contain any (i.e. do not contain detectable amounts) or contain a very low amount (below a predetermined threshold) of endogenous target analytes. For example, if detection of cytokines in blood serum is desired, a standard diluent substantially free of the cytokines can be obtained from screening blood serum samples from various donors. The level of cytokines or other target analytes may vary among the population, and it may be possible to screen a number of donors from the population for the target analytes and thus identify and obtain a biological fluid that is substantially free of target analytes. Screening and selecting such a biological sample would avoid the necessity of removing the target analytes from biological fluids to make a standard diluent.

Whether the standard diluent is obtained by removing target analytes from a biological fluid or by screening biological fluids from donors, a standard diluent is referred to as being “substantially free of two or more different target analytes” when the target analytes are undetectable by immunoassay methods or when the level of the target analytes is lower than a selected sensitivity threshold. For example, depending on the assay to be conducted, the sensitivity level of a target analyte in a sample may variously be selected at less than about, e.g., 20 pg/mL, less than 10 pg/mL, less than 5 pg/mL, or less than 1 pg/mL. In any such case the standard diluent may be said to be “substantially free of two or more different target analytes” when their concentrations are below the selected threshold.

The target analytes can be any components in a biological fluid, such as proteins, peptides, nucleic acids, lipids, carbohydrates, haptens, or combinations thereof. Generally, the target analytes are proteins or peptides. For example, the target analytes can be various cytokines, hormones such as steroids, lipoproteins, glycoproteins, or tumor antigens. In one embodiment, the target analytes are cytokines, such as interleukins, lymphokines, interferons, colony stimulator factors, platelet-activating factors, and/or tumor necrosis factors. In a preferred embodiment, the biological fluid used to make a standard diluent is blood serum, and the target analytes are two or more of interleukin-2 (“IL-2”), interleukin-4 (“IL-4”), interleukin-6 (“IL-6”), interleukin-8 (“IL-8”), interleukin-10 (“IL-10”), granulocyte-macrophage-colony stimulating factor (“GM-CSF”), tumor necrosis factor alpha (“TNF-α”) and interferon gamma (“IFN-γ”).

If necessary, any number of target analytes can be removed, sequentially or simultaneously, from a biological fluid to prepare a standard diluent. For example, two to five hundred target analytes, or two to one hundred target analytes, or two to fifty target analytes, or any integer number in between these ranges can be removed from a biological fluid to make a standard diluent. Typically, between three to thirty, more typically between three to twenty target analytes, even more typically between four to fifteen target analytes are removed from a biological fluid to make a standard diluent. Obviously, only the target analytes that are to be detected in test samples need to be removed from a biological fluid to prepare a standard diluent.

Once a standard diluent substantially free of target analytes is prepared, it can be used to dilute a reference standard (e.g., a concentrated material of target analytes) to make control materials for calibrating the amount of the target analytes in a test sample. In this procedure, generally, a predetermined amount of concentrated material that collectively or separately contains two or more different target analytes is mixed with the standard diluent of the invention. For example, if calibrating IL-2, IL-4, IL-6, IL-8, and IL-10 in a test sample is desired, then a predetermined amount of a concentration material comprising all five interleukins can be added to a standard diluent to make a control material. Then the control materials containing different amounts of target analytes can be used to generate a standard curve that can then be used to quantify the amount of these interleukins in a test sample.

In many cases, it will be useful to prepare a series of control materials at different concentrations of the target analytes, spanning or bracketing the range of concentrations that might be expected in test samples or test samples that are appropriately diluted. For example, a series of control materials may include one control material in which the concentration of the target analytes is approximately equal to that of a patient not suffering from a disease, a second control material containing the target analytes at a concentration substantially higher than that of the first control material, and a third control material containing the target analytes at a concentration substantially higher than those of both the first and second control materials. For example, for detection of cytokines in serum, a series of control materials comprising 5,000 pg/mL, 500 pg/mL, 50 pg/mL 8 pg/mL, 4 pg/mL, 2 pg/mL and 0 pg/mL of cytokines can be used to generate a standard curve.

Accordingly, embodiments of the invention also provide a kit comprising (a) a standard diluent of the invention; and (b) a predetermined amount of one or more concentrated materials that collectively or separately contain two or more different target analytes. Such a kit provides a convenient way for the user to make control materials for an immunoassay, and avoids the need for the user to accurately measure concentrated materials comprising the target analytes. The concentrated materials comprising the target analytes may be in a liquid or solid form. For example, the concentrated material comprising the target analytes may be lyophilized, which can be later dissolved by the user to make control materials.

The kit can further comprise instruction materials for using the standard diluent to produce a series of control materials comprising different concentrations of the target analytes. For example, instructional materials can include how to initiate the dilution series by diluting each concentration material with a standard diluent, together with recommendations for the range of concentrations of target analytes that are expected to be found in a test sample. The kit can also include written instructions for the use of one or more of other reagents in any of the assays described herein. The kit can further include a container containing one or more of the detection reagents with or without labels, and capture reagents, either free or bound to solid supports. Preferably, the kits will also include reagents used in the assays, including reagents useful for detecting the presence of the detectable labels.

In another aspect, the invention also provides a kit for the simultaneous detection of multiple target analytes in a test sample that further comprises solid supports upon which are immobilized capture reagents that bind to the target analytes. In assays using such a kit, solid supports comprising capture reagents that bind to different target analytes can be mixed in a single well (for example), and a test sample can be introduced. The target analytes can be detected using the detection methods known in the art. The amount of each of the multiple target analytes can be determined reliably using the control materials of the present invention. This simultaneous analysis of multiple target analytes from a test sample has obvious cost and convenience benefits. Moreover, only a small amount of a test sample is required since the multiple target analytes can be assayed simultaneously in a single well.

Any suitable capture reagents can be used to bind the target analytes in such kits. For example, capture reagents can be antibodies (e.g., monoclonal antibodies, polyclonal antibodies), antibody fragments, single chain antibodies, etc., that specifically bind to the target analytes. Antibodies will usually bind with a K_{d} of at least about 0.1 mM, more usually at least about 1 μM, preferably at least about 0.1 μM or better, and most preferably, 0.01 μM or better.

Various procedures known in the art can be used for the production of antibodies that specifically bind to target analytes. For the production of polyclonal antibodies, one can use target analytes to inoculate any of various host animals, including but not limited to rabbits, mice, rats, sheep, goats, and the like. Monoclonal antibodies can be prepared by any technique that provides for the production of antibody molecules by continuous cell lines in culture, including the hybridoma technique originally developed by Kohler and Milstein (Nature 256: 495-497 (1975)), as well as the trioma technique, the human B-cell hybridoma technique (Kozbor et al., Immunology Today 4: 72 (1983)), and the EBV-hybridoma technique to produce human monoclonal antibodies (Cole et al. in Monoclonal Antibodies and Cancer Therapy, Alan R. Liss, Inc., pp. 77-96 (1985)).

Fragments of antibodies are also useful as binding moieties. While various antibody fragments can be obtained by the digestion of an intact antibody, those skilled in the art will appreciate that such fragments may be synthesized de novo either chemically or by utilizing recombinant DNA methodology. Single chain antibodies are also useful to construct detection moieties. Methods for producing single chain antibodies were described in, for example, U.S. Pat. No. 4,946,778. Techniques for the construction of Fab expression libraries were described by Huse et al., Science 246: 1275-1281 (1989). These techniques facilitate rapid identification of monoclonal Fab fragments with the desired specificity for target analytes. Suitable binding moieties also include those that are obtained using methods such as phage display.

The capture reagents can be immobilized on the support either by covalent or non-covalent methods, as known in the art. See, e.g., Pluskal et al., BioTechniques 4: 272-283 (1986). Suitable supports include, for example, glasses, plastics, polymers, metals, metalloids, ceramics, organics, and the like. Specific examples include, but are not limited to, microtiter plates, a flat substrate (e.g., a chip), nitrocellulose membranes, nylon membranes, and derivatized nylon membranes, and also microparticles or beads, such as beads of agarose, dextran, and the like. Suitable solid supports and analysis methods for multiplex assays are also described in, e.g., U.S. Pat. Nos. 5,567,627, 5,981,180, 5,641,640, International Application WO98/59360, and Michael et al., Anal. Chem. 70:1242-8 (1998).

In one embodiment, solid supports are a population of microparticles that are classifiable into subgroups, wherein each subgroup is differentiable from others by a differentiation parameter and each subgroup also has capture reagents immobilized thereon which are capable of binding to a different target analyte. A differentiation parameter is a term used herein to denote a distinguishable characteristic that permits separate detection of the assay result in one subgroup from that in another. For example, differentiation parameters that can be used to distinguish among the various subgroups of microparticles include particle size, particle fluorescence, particle light scatter, light emission and absorbance properties. A capture reagent for each target analyte can then be coupled to each subgroup of microparticles. Although different subgroups of microparticles are pooled together in multiplex assays, the differentiation parameter associated with each subgroup of microparticles allows the user to determine which target analyte is bound to which subgroup of microparticles.

In a preferred embodiment, a differentiation parameter that is used to distinguish among various subgroups of microparticles is fluorescence dye or color. For example, the microparticles can have two or more fluorochromes incorporated within them so that microparticles in each subgroup can be differentiated from another based on fluorescence characteristics, such as fluorochrome concentration. Each subgroup of microparticles, for instance, can have different concentrations of a red fluorochrome such as Cy5 together with different concentrations of an orange fluorochrome such as Cy5.5. By varying the concentration of each of the two fluorochromes, hundreds or thousands of subgroups of microparticles with different fluorescent emissions can be obtained. Additional fluorochromes can be incorporated into microparticles to further expand the number of subgroups of microparticles that can be used in a multiplex assays. Microparticles with dyes already incorporated and thereby suitable for use in the present invention are commercially available from suppliers such as Luminex Corporation (Austin, Tex.) and Molecular Probes, Inc. (Eugene, Oreg.).

In another aspect, the kit of the present invention further comprises detection reagents. The presence of target analytes is generally detected using a detection reagent that is composed of a binding moiety that specifically binds to the target analytes. The detection reagents are either directly labeled, i.e., comprise or react to produce a detectable label, or are indirectly labeled, i.e., bind to a molecule comprising or reacting to produce a detectable label. Labels can be directly attached to or incorporated into the detection reagent by chemical or recombinant methods. For example, a label is coupled to a molecule, such as an antibody that specifically binds to a target analyte through a chemical linker.

The detectable labels used in the assays of the present invention, which are attached to the detection reagent, can be primary labels (where the label comprises an element that is detected directly or that produces a directly detectable element) or secondary labels (where the detected label binds to a primary label, e.g., as is common in immunological labeling). An introduction to labels, labeling procedures and detection of labels is found in Polak and Van Noorden (1997) Introduction to Immunocytochemistry, 2nd ed., Springer Verlag, N.Y. and in Haugland (1996) Handbook of Fluorescent Probes and Research Chemicals, a combined handbook and catalogue published by Molecular Probes, Inc., Eugene, Oreg. Patents that described the use of such labels include U.S. Pat. Nos. 3,817,837; 3,850,752; 3,939,350; 3,996,345; 4,277,437; 4,275,149; and 4,366,241.

Primary and secondary labels can include undetected elements as well as detected elements. Useful primary and secondary labels in the present invention can include spectral labels such as green fluorescent protein, fluorescent dyes (e.g., fluorescein and derivatives such as fluorescein isothiocyanate (FITC) and Oregon Green, rhodamine and derivatives (e.g., Texas Red, tetrarhodamine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horseradish peroxidase, alkaline phosphatase etc.), spectral colorimetric labels such as colloidal gold or colored glass or plastic (e.g. polystyrene, polypropylene, latex, etc.) beads. The label can be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

The assays for detecting multiple target analytes can be performed in any of several formats. For example, a sandwich assay can be performed by placing a test sample in contact with solid supports on which are immobilized capture reagents that bind to the target analytes. The capture reagents immobilized on the solid supports are present in excess relative to the suspected quantity range of the target analytes so that all of the target analytes bind. The target analytes, if present in the sample, bind to the capture reagents. The solid supports are then contacted with detection reagents which bind to different epitopes on the target analytes. After incubation of the detected reagents for a sufficient time to bind to the immobilized target analytes, any unbound detection reagents are removed by, e.g., washing. The detectable label (e.g., phycoerythrin) associated with the detection reagents is then detected. For example, if a detectable label is fluorescence, then the fluorescence will be observed in proportion to the amount of the specific target analytes present in the sample.

In another example, competitive binding assays can also be used to detect the target analytes in a sample. The assays are performed by adding labeled analogs of target analytes to a sample. In these assays, the capture reagents bound to the solid supports are present in excess relative to the suspected quantity range of the target analytes so that all of the analytes bind. The labeled analogs and the target analytes present in the sample compete for the binding sites of the capture reagents immobilized on the solid supports. After a suitable incubation period, any remaining unbound analytes and labels are washed away. The amount of labeled analogs of target analytes bound to the solid supports is inversely proportional to the concentration of target analytes in the sample.

The presence of a label can be detected by inspection, or a detector that monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill.

In a preferred embodiment, the solid supports are microparticles and a flow cytometer is used to detect the presence of target analytes in a sample. Methods of and instrumentation for flow cytometry are known in the art. Flow cytometry in general resides in the passage of a suspension of the microparticles as a stream past a light beam and electro-optical sensors, in such a manner that only one microparticle at a time passes through the region. As each microparticle passes this region, the light beam is perturbed by the presence of the microparticle, and the resulting scattered and fluorescent light are detected. The optical signals are used by the instrumentation to identify the subgroup to which each microparticle belongs, along with the presence and amount of label, so that individual assay results are achieved. Descriptions of instrumentation and methods for flow cytometry are found in, e.g., McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544.

As an illustration, antibodies that bind to target analyte A are immobilized to a subgroup of microparticles comprising a green fluorochrome; antibodies that bind to target analyte B are immobilized to a subgroup of microparticles with a red fluorochrome; and antibodies that bind to target analyte C are immobilized to a subgroup of microparticles with a yellow fluorochrome. After coupling the microparticles in each subgroup with their respective antibodies, then they are pooled together and a sample is applied. Then the detection reagents that bind to the target analytes are added to the mixture. Generally, the detection reagent is labeled with another measurable label, which is distinguishable from fluorochromes contained in the microparticles. Then, the capture reagents and the detection reagents form a sandwich around the target analytes. Then, this binding can be visualized by the label (e.g., phycoerythrin) associated with the detection reagents.

The microparticles are then run through a flow cytometer, and each microparticle is classified by its distinguishing characteristics. The presence of target analytes specific for capture reagents can be detected by measuring phycoerythrin of each microparticle. The difference in parameters of microparticles, such as size or color, allows one to determine the subgroup to which a microparticle belongs, which serves as an identifier for the capture reagents carried on the microparticles. The parameter from phycoerythrin of the microparticles indicates the extent to which the target analytes are reactive with the capture reagents are present in a test sample. These multiplex analysis methods are described in detail in, e.g., U.S. Pat. Nos. 5,981,180 and 5,567,637. The above-described multiplex assays are only illustrations of the use of the invention; those of ordinary skill in the art will recognize that standard diluents of the present invention may be used in other multiplex assay formats as well.

The following examples are illustrative of the invention, but are in no way intended to limit the scope thereof.

### EXAMPLE 1

This example illustrates the preparation of a standard diluent according to this invention from a human serum, and its use in a process for determining eight cytokine analytes in a serum sample.

Preparation of Standard Diluent:

An affinity chromatographic column was prepared to substantially remove the eight target analytes from a serum sample as follows.

The target analytes were IL-2, IL-4, IL-6, IL-8, IL-10, GM-SCF, IFNγ and TNFα. Monoclonal antibodies specific to each target analyte were obtained, purified of salt, and stored on ice.

Eight columns were constructed, one for separation of each target analyte using the monoclonal antibody specific for it. Slurries of Affi-gel® beads were prepared, in 100-μL portions, each containing 50 μL of beads. These beads contain a neutral 10-atom spacer arm that allows for covalent coupling of the antibodies to the beads. The slurries were washed with deionized water and placed in the columns. Five μg of each antibody was added to a column; the columns were then incubated for 4-16 hours at 40° C. on a rotating shaker. The beads were then washed with phosphate buffered saline.

Human blood serum was then passed through the eight columns in series. The serum, now substantially free of the eight target analytes, was collected for use as a standard diluent in the following assay.

Assay for Target Analytes

Each patient's blood sample was allowed to clot; then it was centrifuged and the serum collected. Diluted samples were prepared by diluting portions of the serum with the standard diluent prepared above, in a ratio of 3 volumes diluent per volume sample.

Cytokine standards were prepared from lyophilized cytokines reconstituted with sterile distilled water to produce standard stocks continuing 500,000 pg/mL of the respective cytokine. Each standard stock was then diluted to produce a series of diluted standards having concentrations of 50,000, 5000, 500, 50, 8, 4, 2 and 0 pg/mL of the cytokine question, respectively.

For determination of the target analytes, eight groups of 5.5-micron polystyrene beads were selected, with each group of beads characterized by a different fluorochrome. Monoclonal antibodies specific to the eight target analytes were each covalently coupled to beads containing a particular fluorochrome. A total of 2.6×10⁶ beads/mL per target analyte was prepared.

A 96-well plate was prepared by pre-wetting with an assay buffer. One well was utilized for each patient sample, with one or more additional wells being utilized for control(s). Each well was filled with 50 μL of materials, including 2 μL of each of the eight bead-antibody conjugates (i.e., a total of 16 μL of conjugated beads) and the remainder Bio-Rad Bio-Plex® assay buffer (in this case, 34 μL of buffer). A combined bead stock was prepared, with sufficient volume as needed for the total number of wells in the test (50 μL bead stock per well), and was vortexed before being added to the wells.

The filled plate was covered, then shaken at 1100 rpm, for 30 seconds, followed by 300 rpm, for 30 minutes. (If desired, sensitivity of the test may be improved by overnight incubation at 4° C. with shaking at 300 rpm.) The beads were washed three times with Bio-Plex buffer. The plate was placed in the dark.

A stock of a series of biotinylated detection antibodies specific to the eight cytokine antibodies was prepared. The detection antibody stock (100×) was diluted with Bio-Rad Bio-Plex® detection antibody diluent to provide a final volume of 50 μL diluted detection antibody per well, with each well receiving 0.5 μL of each detection antibody.

The diluted detection antibody stock was vortexed, then 50 μL was added to each well. The plate was covered and shaken at room temperature for 30 seconds at 1100 rpm, then for 30 minutes at 300 rpm. The beads were again washed three times with the wash buffer. The plate was dried and again kept in the dark.

A streptavidin-phycoerytherin conjugate was then coupled to the bead-antibody conjugates. The streptavidin-phycoerytherin conjugatae (original concentration 100×) was diluted to 1× with the assay buffer; then 50 μL of the diluted conjugate was vortexed and added to each well. The plate was again covered, shaken and washed. The beads in each well were resuspended in the assay buffer, shaken and kept in the dark until the samples were read.

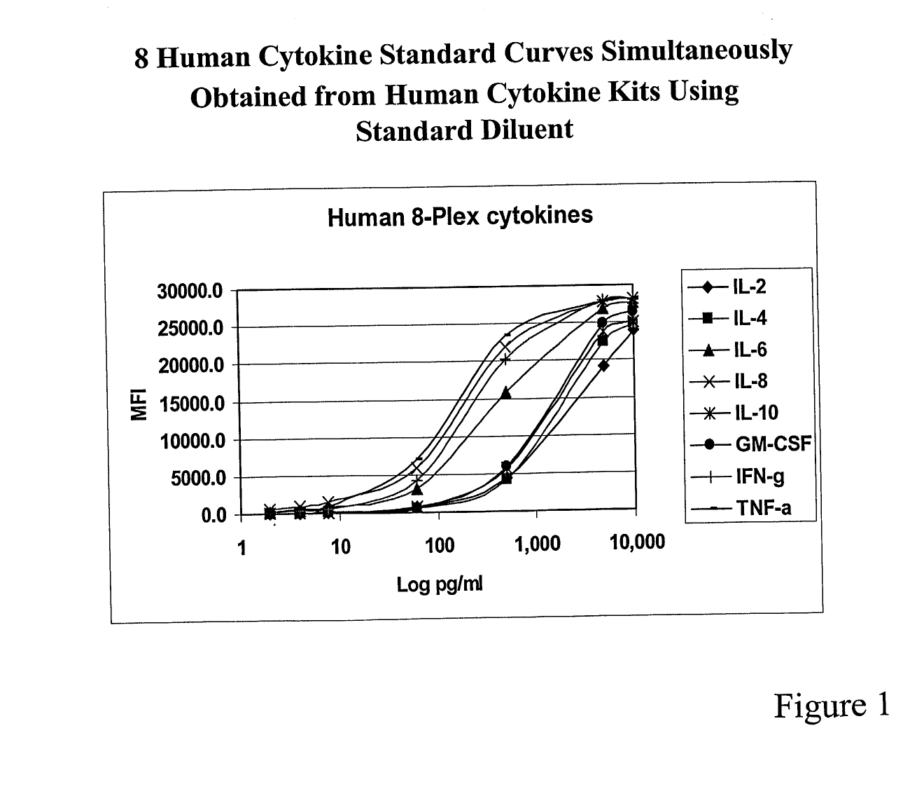

The eight cytokines in the patient samples and controls were determined using a flow cytometer. The results are shown in the following Table 1. Standard curves are shown in FIG. 1.

TABLE 1
Patient Serum Samples Screened By 8-Plex Cytokine Assays
(Cytokine concentrations given in pg/mL)

| | | | IL- | | | | | |
|---|---|---|---|---|---|---|---|---|
| Patient | IL-2 | IL-4 | 10 | GM-CSF | IFNγ | TNFα | IL-8 | IL-6 |
| F38 | 0 | 0 | 0 | 0 | 0 | 0 | 0 | 0 |
| F45 | 50 | 111 | 70 | 300 | 822 | 1200 | 58 | 1815 |
| F49 | 0 | 0.1 | 58 | 0.1 | 0 | 0.1 | 0 | 0.5 |
| F51 | 0 | 0.2 | 0 | 0 | 0.5 | 0.1 | 0 | 0.5 |
| F54 | 40 | 96 | 60 | 285 | 741 | 1100 | 47 | 1798 |
| F59 | 0 | 11 | 10 | 250 | 170 | 734 | 0 | 15 |
| M61 | 0 | 7.2 | 8 | 250 | 144 | 668 | 0 | 3.5 |
| M67 | 0.1 | 1.5 | 10 | 11 | 12 | 10 | 0 | 4 |
| M71 | 0.5 | 77 | 11 | 150 | 963 | 1257 | 1.5 | 337 |
| M74 | 0.5 | 40 | 8 | 150 | 928 | 1215 | 1.5 | 339 |
| M75 | 0.5 | 72 | 5 | 145 | 926 | 1077 | 1 | 355 |

M = Male
F = Female

### EXAMPLE 2

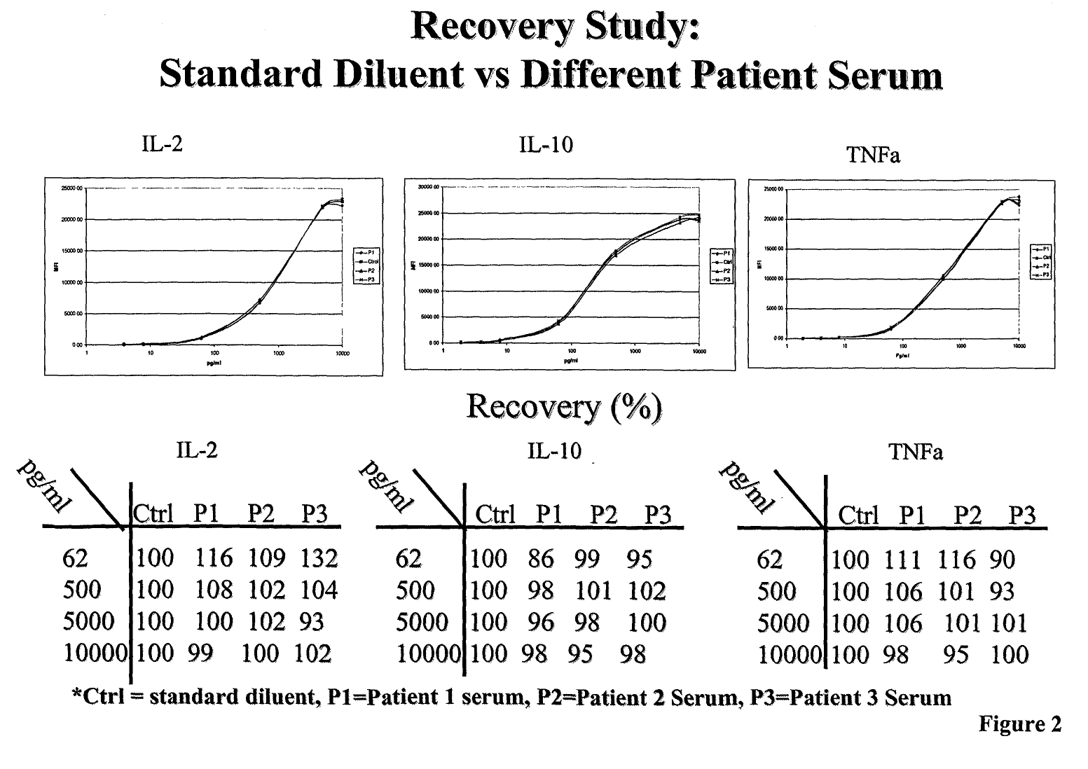

This example illustrates a recovery assay including a standard diluent according to this invention and sera from three different patients. Cytokines determined were IL-2, IL-10 and TNFA. Amounts of from 0-10,000 pg/mL of antigens were spiked into 50 μl of standard diluent prepared according to this invention (“control”) and lacking the three target cytokines, and into 1:4 dilutions of the three patient sera. Samples were run in duplicate. The results are shown in FIG. 2.

### EXAMPLE 3

This example illustrates the use of a standard diluent according to the invention for determination of cytokines in mouse sera in a multiplex assay.

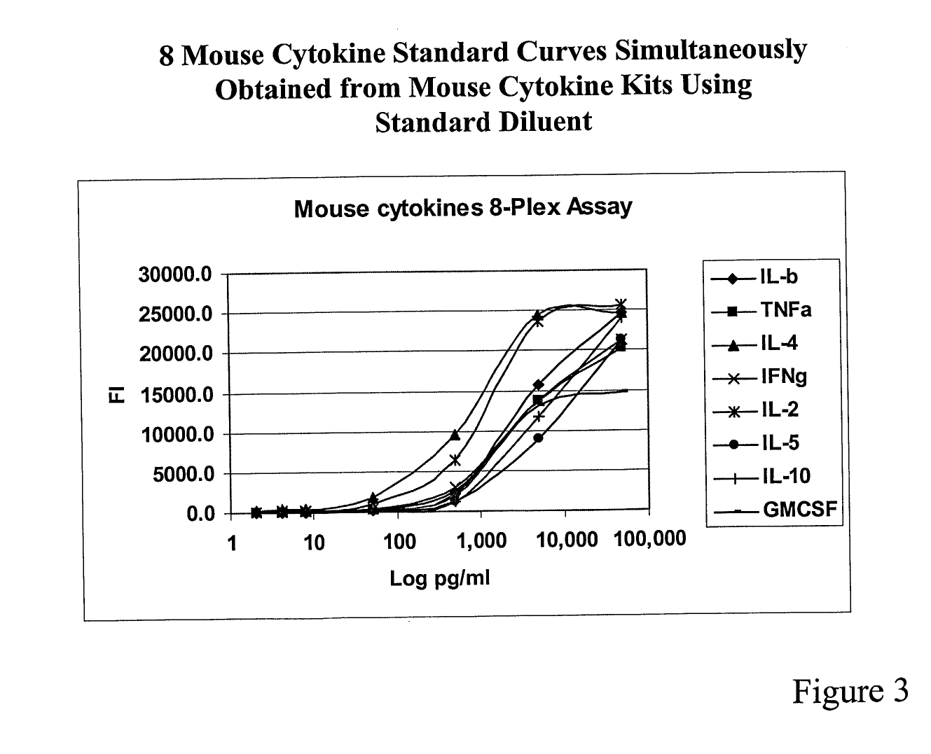

Similarly to Example 1, mice sera were tested for the same eight cytokines. The standard curves determined in this test are shown in FIG. 3. EXAMPLE 4

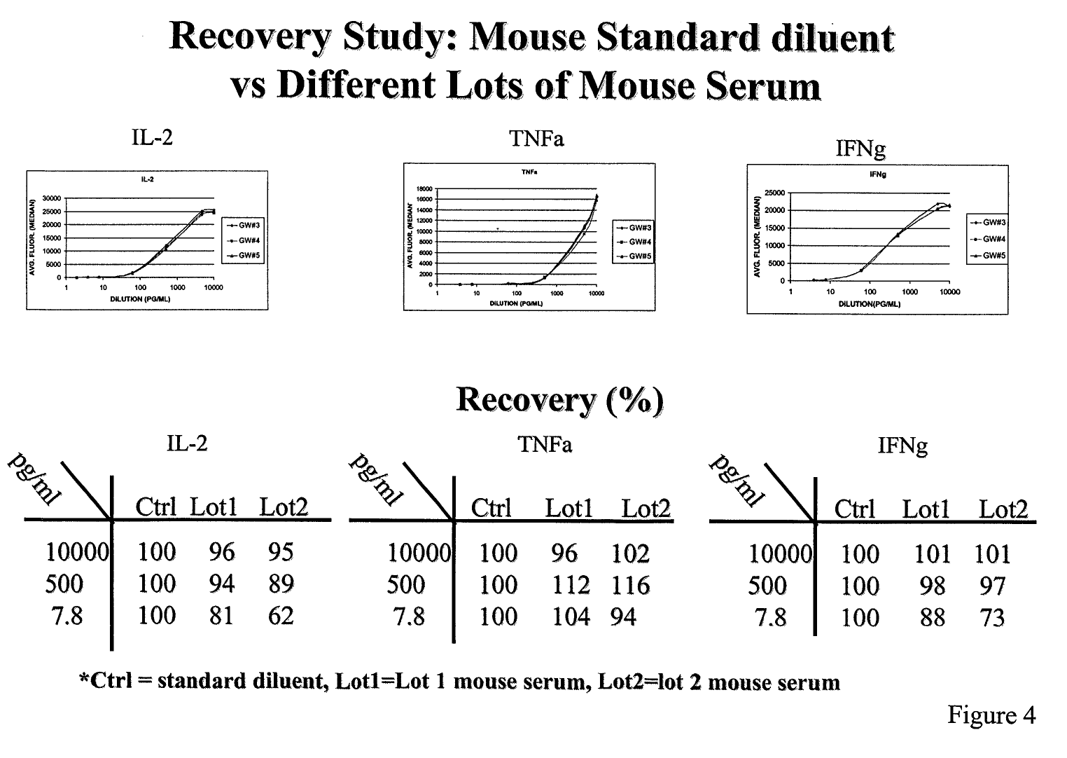

This example illustrates a recovery assay in mice. The procedure was similar to that of Example 2, except that 0-50,000 pg/mL of antigens was spiked into 50 μL of a standard dilution of three different batches of pooled mouse sera. The results are shown in FIG. 4.

The present invention provides standard diluents for multiplex assays, kits and methods relating to the standard diluent. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

What is claimed is:

1. A kit comprising:
(a) a standard diluent comprising a biological fluid normally including two or more different target analytes but substantially free of the two or more different target analytes; and
(b) a predetermined amount of one or more concentrated materials that collectively or separately contain the two or more different target analytes.

2. The kit in accordance with claim 1 in which the standard diluent is produced by removing the two or more different target analytes from the biological fluid by affinity chromatography.

3. The kit in accordance with claim 1 in which the standard diluent is obtained from a biological fluid of a host having the biological fluid substantially free of the two or more different target analytes.

4. The kit in accordance with claim 2 in which the affinity chromatography comprises removing the two or more different target analytes using antibodies that bind to the target analytes.

5. The kit in accordance with claim 1 in which the biological fluid is selected from the group consisting of serum, plasma, urine, cerebrospinal fluid, cell extracts, amniotic fluid, sweat, tear, saliva or nasal secretions.

6. The kit in accordance with claim 5 in which the biological fluid is obtained from human or mouse.

7. The kit in accordance with claim 1 in which the two or more different target analytes are cytokines.

8. The kit in accordance with claim 7 in which the cytokines are selected from interleukins, lymphokines, interferons, colony stimulator factors, platelet-activating factors, and/or tumor necrosis factors.

9. The kit in accordance with claim 1 in which the target analytes are two or more of IL-2, IL-4, IL-6, IL-8, IL-10, GM-CSF, TNF-α and IFN-γ.1

10. The kit in accordance with claim 1 in which the two or more different target analytes are mixed together to form a single concentrated material in part (b).

11. The kit in accordance with claim 1, the kit further comprising instruction materials for using the standard diluent to produce a series of control materials comprising different concentrations of the target analytes.

12. The kit in accordance with claim 1, the kit further comprising solid supports having immobilized thereon capture reagents that bind to the target analytes.

13. The kit in accordance with claim 12 in which the solid supports are classifiable into subgroups, each subgroup differentiable from others by a differentiation parameter and each subgroup having immobilized thereon a capture reagent capable of binding to a different target analyte.

14. The kit in accordance with claim 13 in which the differentiation parameter is color or fluorescence of the solid supports.

15. The kit in accordance with claim 12 in which the solid supports are microparticles.

16. The kit in accordance with claim 12 in which the capture reagents are antibodies that bind to the target analytes.

17. The kit in accordance with claim 16, the kit further comprising detection reagents that bind to the target analytes.

18. A control material for calibrating the amount of two or more different target analytes in a test sample in an immunoassay, the control material comprising:
(a) a predetermined amount of a concentrated material comprising the two or more different target analytes mixed with (b) a standard diluent comprising a biological fluid normally including the two or more different analytes but substantially free of the two or more different target analytes.

19. The control material in accordance with claim 18 in which the target analytes are cytokines.

20. A kit for detecting two or more different target analytes in a serum or plasma sample, the kit comprising:
(a) solid supports that are classifiable into subgroups, each subgroup differentiable from others by a differentiation parameter and each subgroup capable of having immobilized thereon a capture reagent that binds to a different target analyte; and
(b) a standard diluent comprising serum or plasma that is substantially free of the two or more different target analytes.

21. The kit in accordance with claim 20, wherein the differentiation parameter is color or fluorescence of the solid supports.

22. The kit in accordance with claim 20 in which the solid supports are microparticles.

23. The kit in accordance with claim 20 in which the capture reagent for each target analyte is immobilized on each subgroup of the solid supports.

24. The kit in accordance with claim 20 in which the standard diluent is produced by removing the two or more different target analytes from the serum or plasma by affinity chromatography.

25. The kit in accordance with claim 20 in which the standard diluent is obtained from a host's serum or plasma which has an undetectable endogenous level of the two or more different target analytes.

26. The kit in accordance with claim 20 in which the serum or plasma for the standard diluent is obtained from human or mouse.

27. The kit in accordance with claim 20 in which the two or more different target analytes are cytokines.

28. The kit in accordance with claim 27 in which the cytokines are selected from interleukins, lymphokines, interferons, colony stimulator factors, platelet-activating factors, and/or tumor necrosis factors.

29. The kit in accordance with claim 27 in which the target analytes are two or more of IL-2, IL-4, IL-6, IL-8, IL-10, GM-CSF, TNF-α, and INF-γ.

30. The kit in accordance with claim 20, the kit further comprising a predetermined amount of one or more concentrated materials that collectively or separately contain the two or more different target analytes.

31. The kit in accordance with claim 20, the kit further comprising detection reagents that bind to the target analytes.

32. A method of conducting a simultaneous assay for two or more target analytes in which a standard diluent is used to dilute one or more reference standards, the method comprising using as the standard diluent a biological fluid substantially free of the two or more target analytes.

33. The method in accordance with claim 32 in which the assay is conducted for the target analytes in a first biological fluid, and the diluent comprises a second biological fluid comprising essentially the same matrix components as the first biological fluid, the second biological fluid being substantially free of the two or more target analytes.

34. The method in accordance with claim 33 in which the second biological fluid is obtained by screening a series of biological fluids and identifying one or more biological fluids containing the two or more target analytes at a concentration below a predetermined threshold.

35. The method in accordance with claim 33 in which the second biological fluid is obtained by treating a biological fluid to remove the target analytes so as to decrease the concentrations thereof to concentrations below predetermined thresholds.

36. The method in accordance with claim 35 in which the target analytes are removed by affinity chromatography.

37. The method in accordance with claim 36 in which the target analytes are removed by contacting the biological fluid with antibodies that bind to the target analytes.

38. The method in accordance with claim 33 in which the biological fluid is selected from interleukins, lymphokines, interferons, colony stimulator factors, platelet-activating factors, and/or tumor necrosis factors.

39. The method in accordance with claim 33 in which the two or more different target analytes are cytokines.

40. The method in accordance with claim 33 in which the cytokines are selected from interleukins, lymphokines, interferons, colony stimulator factors, platelet-activating factors, and/or tumor necrosis factors.

41. The method in accordance with claim 40 in which the target analytes are two or more of IL-2, IL-4, IL-6, IL-8, IL-10, GM-CSF, TNF-α and/or INF-γ.

42. A method of preparing a standard diluent for use in a simultaneous assay for two or more target analytes, comprising treating a biological fluid containing the target analytes to remove the target analytes so as to decrease the concentrations thereof to concentrations below predetermined thresholds.

43. The method in accordance with claim 42 in which the target analytes are removed by affinity chromatography.

44. The method in accordance with claim 43 in which the target analytes are removed by contacting the biological fluid with antibodies that bind to the target analytes.

45. The method in accordance with claim 42 in which the biological fluid is selected from interleukins, lymphokines, interferons, colony stimulator factors, platelet-activating factors, and/or tumor necrosis factors.

46. The method in accordance with claim 42 in which the two or more different target analytes are cytokines.

47. The method in accordance with claim 46 in which the cytokines are selected from interleukins, lymphokines, interferons, colony stimulator factors, platelet-activating factors, and/or tumor necrosis factors.

48. The method in accordance with claim 47 in which the target analytes are two or more of IL-2, IL-4, IL-6, IL-8, IL-10, GM-CSF, TNF-α and/or INF-γ.
